(19) United States
(12) Patent Application Publication
(10) Pub. No.: US 2022/0299381 A1
(43) Pub. Date: Sep. 22, 2022

(54) STRESS ANALYSIS SYSTEM

(71) Applicant: SHUN-TIEN HUNG, Taichung City (TW)

(72) Inventor: SHUN-TIEN HUNG, Taichung City (TW)

(21) Appl. No.: 17/697,528

(22) Filed: Mar. 17, 2022

(30) Foreign Application Priority Data
Mar. 18, 2021 (TW) 110109843

(51) Int. Cl. G01L 1/00; G01L 5/16

(52) U.S. Cl. CPC G01L 1/005; G01L 5/16

(57) ABSTRACT

A stress analysis system for performing analysis of a force exerted on a test object is provided, wherein the test object is defined to have a plurality of predetermined areas at different positions thereof. The stress analysis system includes: an analysis module, and at least two stress bearing modules separate from each other, each of which is located at one of the predetermined areas. Each of the stress bearing modules includes a supporting strip component and a feature sensor, wherein the supporting strip component is able to produce a stressed feature in at least one spatial dimension when being subjected to the force, and the feature sensor is able to sense a stress state of the corresponding predetermined area of the test object in at least one spatial dimension, such that the stress analysis system can accurately analyze the stress state of the test object.

### CROSS-REFERENCE TO RELATED APPLICATIONS

This application claims the priority of Republic of China Patent Application No. 110109843 filed on Mar. 18, 2021, in the State Intellectual Property Office of the R.O.C., the disclosure of which is incorporated herein by reference.

### BACKGROUND OF THE INVENTION

### Field of the Invention

The present invention relates to a stress analysis system, and more particularly, a stress analysis system for performing analysis of an external force acting on a test object in at least one spatial dimension, by sensing elastic deformation in at least one spatial dimension of a supporting strip component on which the force is exerted.

### Descriptions of the Related Art

Various stress bearing products are available in our daily life, which are usually subjected to analysis of their stress states when they are in use. Pillow is a common example of the stress bearing products, for supporting a user's head whose posture lying on a pillow greatly affects his/her sleep quality. The only way to know the user's posture lying on the pillow is by analyzing a stress state of the pillow.

However, the stress bearing products structurally have an adverse impact on the number and positions of sensors being installed for sensing the stress states of the stress bearing products, thereby making their stress states not able to be accurately analyzed. Therefore, how to optimize installation of the sensors to improve accuracy of stress state analysis is an important task to be solved in the art.

### SUMMARY OF THE INVENTION

In view of the above drawbacks in the prior art, a primary object of the present invention is to provide a stress analysis system for performing analysis of a force exerted on a test object, the test object being defined to have at least two predetermined areas at different positions thereof, the stress analysis system including: at least two stress bearing modules separate from each other, each of which corresponds to one of the at least two predetermined areas on the test object, each of the stress bearing modules including a supporting strip component and a feature sensor, wherein the supporting strip component is able to produce a stressed feature for its corresponding one of the predetermined areas when being subjected to the force, and the feature sensor is able to sense the stressed feature from the supporting strip component to obtain a sensing result; and an analysis module for obtaining an effect on the supporting strip component from the force exerted on the test object according to the sensing result from the feature sensor of each of the stress bearing modules so as to analyze a stress state of each of the predetermined areas of the test object.

Preferably, in the stress analysis system said above, the feature sensor is Gyro, wherein Gyro is able to sense a displacement feature of the supporting strip component, with the displacement feature used as the stressed feature.

Preferably, in the stress analysis system said above, the feature sensor is acceleration sensor, wherein the acceleration sensor is able to sense an acceleration feature of the supporting strip component, with the acceleration feature used as the stressed feature.

Preferably, in the stress analysis system said above, the feature sensor is strain sensor, wherein the strain sensor is able to sense a strain feature of the supporting strip component, with the strain feature used as the stressed feature.

Preferably, in the stress analysis system said above, the predetermined areas include a left area and a right area, and the stress bearing modules include a left stress bearing module and a right stress bearing module, wherein, the left stress bearing module is provided at the left area, and has a left supporting strip component and a left strain sensor, wherein the left supporting strip component provides elastic support for the left area, allowing the force exerted on the test object to cause left elastic deformation of the left supporting strip component, and the left strain sensor is used to sense the left elastic deformation; and the right stress bearing module is provided at the right area, and has a right supporting strip component and a right strain sensor, wherein the right supporting strip component provides elastic support for the right area, allowing the force exerted on the test object to cause right elastic deformation of the right supporting strip component, and the right strain sensor is used to sense the right elastic deformation; the analysis module is able to receive sensing results from the left strain sensor and the right strain sensor respectively to obtain the degree of left elastic deformation and the degree of right elastic deformation, and to analyze a stress state of the left area and a stress state of the right area respectively according to the degree of left elastic deformation and the degree of right elastic deformation; wherein, when the degree of left elastic deformation is greater than the degree of right elastic deformation, the analysis module determines that a main position of the test object being subjected to the force is close to the left area; and when the degree of right elastic deformation is greater than the degree of left elastic deformation, the analysis module determines that a main position of the test object being subjected to the force is close to the right area.

Preferably, in the stress analysis system said above, the left supporting strip component has a left longitudinal elastic supporting structure and a left transverse elastic supporting structure, wherein the left longitudinal elastic supporting structure provides longitudinal elastic support for the left area, allowing the force exerted on the test object to cause left longitudinal elastic deformation of the left longitudinal elastic supporting structure, and wherein the left transverse elastic supporting structure provides transverse elastic support for the left area, allowing the force exerted on the test object to cause left transverse elastic deformation of the left transverse elastic supporting structure, wherein the left strain sensor senses the degree of left longitudinal elastic deformation and the degree of left transverse elastic deformation by the left supporting strip component, and the analysis module analyzes a longitudinal stress state and a transverse stress state of the left area respectively according to the degree of left longitudinal elastic deformation and the degree of left transverse elastic deformation.

Preferably, in the stress analysis system said above, the left longitudinal elastic supporting structure is connected to the left transverse elastic supporting structure to form a left space, wherein the left space is able to receive the left strain sensor or the analysis module, and accommodate the left longitudinal elastic deformation of the left longitudinal elastic supporting structure or the left transverse elastic deformation of the left transverse elastic supporting structure.

Preferably, in the stress analysis system said above, the test object is a pillow, the left space provides ventilation for the pillow, and the left longitudinal elastic supporting structure and the left transverse elastic supporting structure respectively provide longitudinal elastic support and transverse elastic support for a left part of the pillow.

Preferably, in the stress analysis system said above, the right supporting strip component has a right longitudinal elastic supporting structure and a right transverse elastic supporting structure, wherein the right longitudinal elastic supporting structure provides longitudinal elastic support for the right area, allowing the force exerted on the test object to cause right longitudinal elastic deformation of the right longitudinal elastic supporting structure, and wherein the right transverse elastic supporting structure provides transverse elastic support for the right area, allowing the force exerted on the test object to cause right transverse elastic deformation of the right transverse elastic supporting structure, wherein the right strain sensor senses the degree of right longitudinal elastic deformation and the degree of right transverse elastic deformation by the right supporting strip component, and the analysis module analyzes a longitudinal stress state and a transverse stress state of the right area respectively according to the degree of right longitudinal elastic deformation and the degree of right transverse elastic deformation.

Preferably, in the stress analysis system said above, the right longitudinal elastic supporting structure is connected to the right transverse elastic supporting structure to form a right space, wherein the right space is able to receive the right strain sensor or the analysis module, and accommodate the right longitudinal elastic deformation of the right longitudinal elastic supporting structure or the right transverse elastic deformation of the right transverse elastic supporting structure.

Preferably, in the stress analysis system said above, the test object is a pillow, the right space provides ventilation for the pillow, and the right longitudinal elastic supporting structure and the right transverse elastic supporting structure respectively provide longitudinal elastic support and transverse elastic support for a right part of the pillow.

Preferably, in the stress analysis system said above, the predetermined areas further include a middle area, and the stress bearing modules further include at least one middle stress bearing module, wherein, the middle stress bearing module is provided at the middle area, and has a middle supporting trip component and a middle strain sensor, wherein the middle supporting strip component provides elastic support for the middle area, allowing the force exerted on the test object to cause middle elastic deformation of the middle supporting strip component, and the middle strain sensor is used to sense the middle elastic deformation; the analysis module is further able to receive a sensing result from the middle strain sensor to obtain the degree of middle elastic deformation, and to analyze a stress state of the middle area according to the degree of middle elastic deformation, wherein, when the degree of middle elastic deformation is respectively greater than the degree of right elastic deformation and the degree of left elastic deformation, the analysis module determines that a main position of the test object being subjected to the force is close to the middle area.

Preferably, in the stress analysis system said above, the middle supporting strip component has a middle longitudinal elastic supporting structure and a middle transverse elastic supporting structure, wherein the middle longitudinal elastic supporting structure provides longitudinal elastic support for the middle area, allowing the force exerted on the test object to cause middle longitudinal elastic deformation of the middle longitudinal elastic supporting structure, and wherein the middle transverse elastic supporting structure provides transverse elastic support for the middle area, allowing the force exerted on the test object to cause middle transverse elastic deformation of the middle transverse elastic supporting structure, wherein the middle strain sensor senses the degree of middle longitudinal elastic deformation and the degree of middle transverse elastic deformation by the middle supporting strip component, and the analysis module analyzes a longitudinal stress state and a transverse stress state of the middle area respectively according to the degree of middle longitudinal elastic deformation and the degree of middle transverse elastic deformation.

Preferably, in the stress analysis system said above, the middle longitudinal elastic supporting structure is connected to the middle transverse elastic supporting structure to form a middle space, wherein the middle space is able to receive the middle strain sensor or the analysis module, and accommodate the middle longitudinal elastic deformation of the middle longitudinal elastic supporting structure or the middle transverse elastic deformation of the middle transverse elastic supporting structure.

Preferably, in the stress analysis system said above, the test object is a pillow, the middle space provides ventilation for the pillow, and the middle longitudinal elastic supporting structure and the middle transverse elastic supporting structure respectively provide longitudinal elastic support and transverse elastic support for a middle part of the pillow.

Preferably, in the stress analysis system said above, the middle supporting strip component, the left supporting strip component and the right supporting strip component are each made of a composite material that is selected from a group consisting of carbon fiber and glass fiber.

In summary, the stress analysis system of the present invention is for performing analysis of a force exerted on a test object, wherein the test object is defined to have a plurality of predetermined areas at different positions thereof. The stress analysis system includes: an analysis module, and at least two stress bearing modules separate from each other, each of which is located at one of the predetermined areas. Each of the stress bearing modules includes a supporting strip component and a feature sensor, wherein the supporting strip component is able to produce a stressed feature in at least one spatial dimension when being subjected to the force, and the feature sensor is able to sense a stress state of the corresponding predetermined area of the test object in at least one spatial dimension, such that the stress analysis system uses a small number of sensors but can accurately analyze the stress state of the test object, thereby solving undesirable problems encountered in the art.

### BRIEF DESCRIPTION OF THE DRAWINGS

The above and other aspects, features and other advantages of the present invention will be more clearly understood from the following detailed description taken in conjunction with the accompanying drawings, in which:

FIGS. 1 to 3 are schematic diagrams showing a stress analysis system according to the present invention being applied to a test object.

FIG. 4 is a schematic diagram showing the stress analysis system according to the present invention at a first angle of view.

FIG. 5 is a schematic diagram showing the stress analysis system according to the present invention respectively at a second angle of view.

FIG. 6 is a usage status diagram of the stress analysis system according to the present invention.

FIG. 7 is a schematic diagram showing deformation of left stress bearing module according to the present invention.

FIG. 8 is a cross-sectional diagram of component shown in FIG. 7 cut along line AA.

FIG. 9 is a schematic diagram showing deformation of middle stress bearing module according to the present invention.

FIG. 10 is a cross-sectional diagram of component shown in FIG. 9 cut along line BB.

FIG. 11 is a schematic diagram showing deformation of right stress bearing module according to the present invention.

FIG. 12 is a cross-sectional diagram of component shown in FIG. 11 cut along line CC.

FIG. 13 is a schematic diagram of a supporting strip component according to the present invention.

### DETAILED DESCRIPTION OF THE PREFERRED EMBODIMENT

Embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In the drawings, the shapes and dimensions of elements may be exaggerated for clarity, and the same reference numerals will be used throughout to designate the same or like components.

The following content will be combined with the drawings to illustrate the technical content of the present invention through specific embodiments. Those familiar with the technology can easily understand the other advantages and effects of the present invention from the content discussed in this specification. This invention can also be implemented or applied by other different specific embodiments. Various details in this specification can also be modified and changed based on different viewpoints and inventions without departing from the spirit of this invention. Especially, the quantitative relationship, proportional relationship, and relative position of each component in the drawings are only exemplary, and do not represent the actual implementation of this invention.

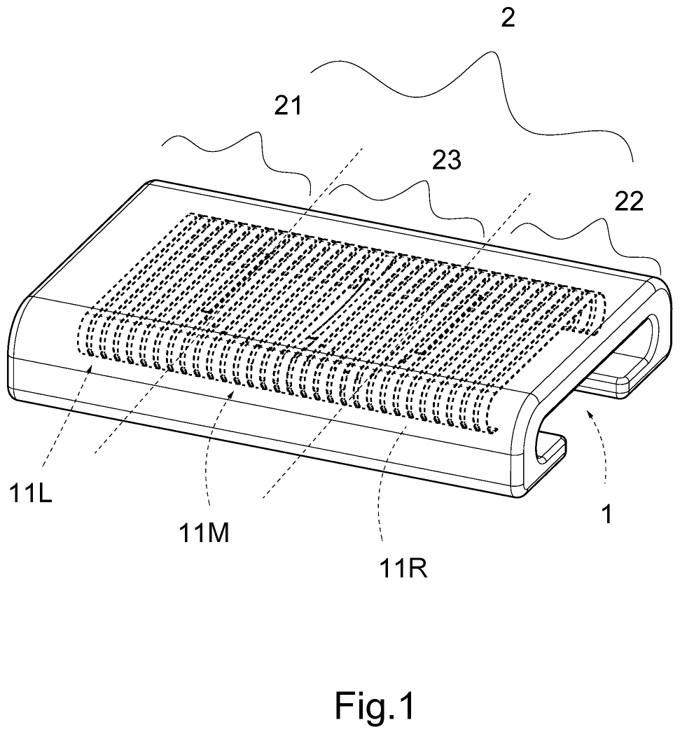

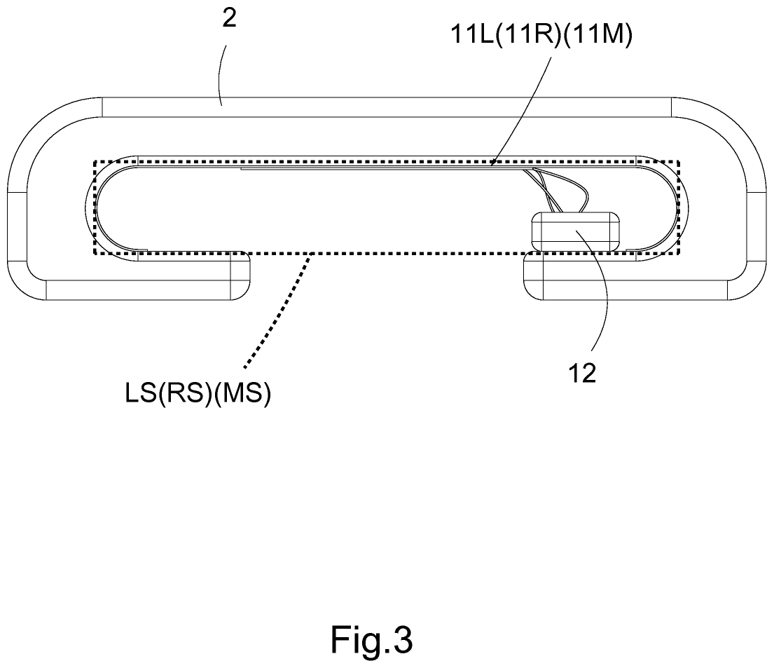

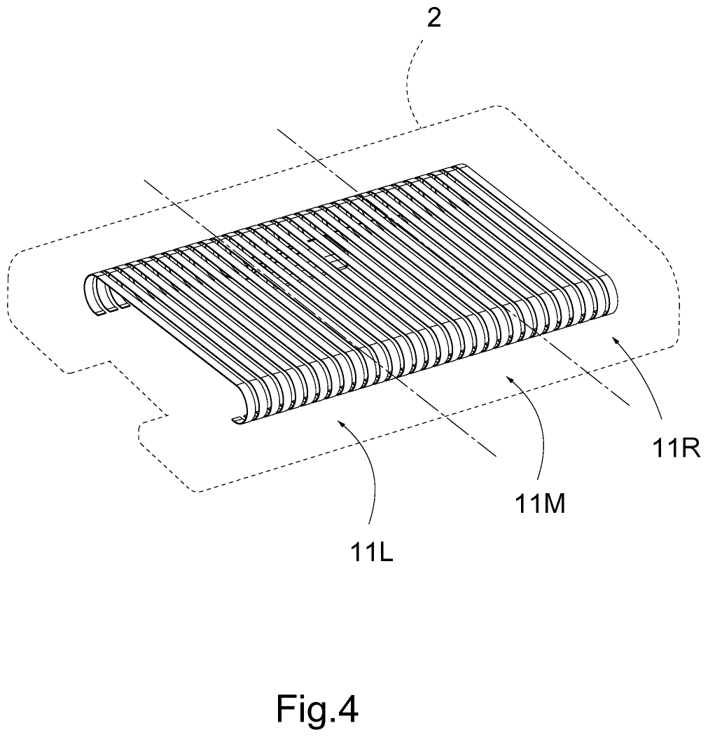

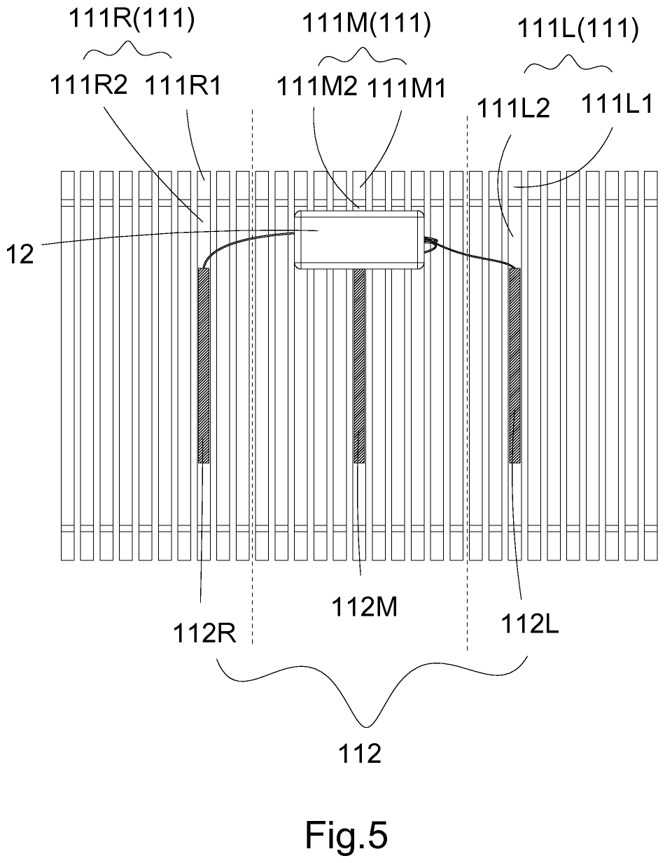

Technical features of the present invention are disclosed as follows with reference to FIGS. 1 to 13.

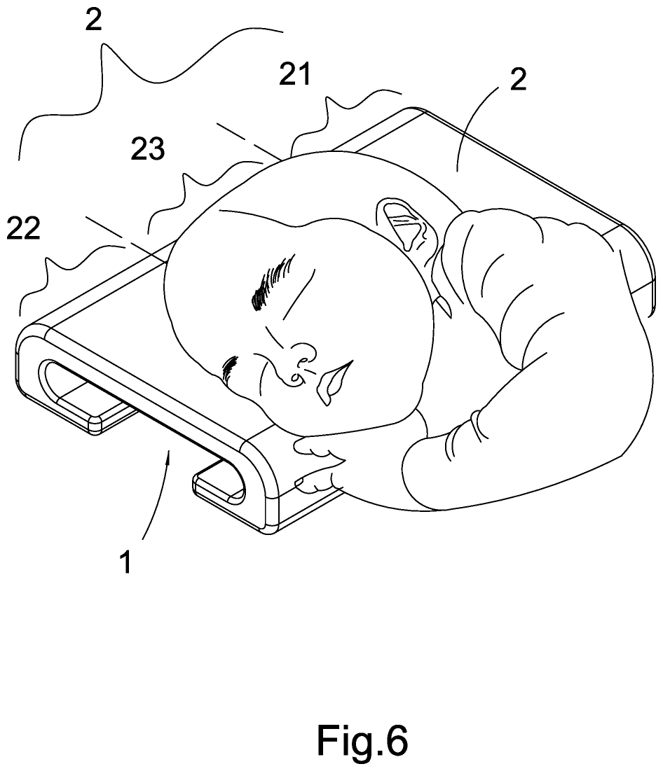

Referring to FIGS. 1 to 6, the present invention provides a stress analysis system 1 for performing stress analysis on a test object 2 to determine a stress state of the test object 2 so as for therapeutic or other purposes. The test object 2 is a stress bearing product capable of sustaining a force from an external subject (such as chair or bed), and is, for example, a pillow for supporting weight of a user's head as shown in FIG. 6.

In the present invention, the test object 2 is defined to have at least two predetermined areas at different locations thereon, and the stress analysis system 1 includes an analysis module 12 and at least two stress bearing modules 11. The stress bearing modules 11 are separate from each other, and each of them corresponds to one of the at least two predetermined areas of the test object 2. Each of the stress bearing modules 11 includes a supporting strip component 111 and a feature sensor 112, wherein the feature sensor 112 is mounted on the supporting strip component 111. The supporting strip component 111 is used to produce a stressed feature for its corresponding predetermined area on the test object 2 when being subjected to an external force, and the feature sensor 112 is used to sense the stressed feature produced by the supporting strip component 111 so as to obtain a stressed feature sensing result of the supporting strip component 111 of each of the stress bearing modules 11. As the feature sensor 112 is not directly installed at the test object 2, its number and location are not physically restricted by the test object 2.

The analysis module 12 is connected to all the feature sensors 112 of the stress bearing modules 11, and is used to receive the stressed feature sensing result of each of the supporting strip components 111, so as to analyze a stress state of each of the predetermined areas on the test object 2 and further to determine if the test object 2 is in an expected stressed condition (such as position or strength of the force acting on the test object 2). This allows each of the feature sensors 112 to sense a stressed feature produced by a corresponding one of the supporting strip components 111 in at least one spatial dimension and thus to sense a stress state of a corresponding one of the predetermined areas on the test object 2 in at least one spatial dimension, thereby reducing the number of sensors required for accurately sensing a stress state of a stress bearing product (that is, the test object 2).

With the above configuration that each of the predetermined areas on the test object 2 is provided with one stress bearing module 11, the analysis module 12 can then sense and analyze the stress state of each of the predetermined areas on the test object 2 by each corresponding stress bearing module 11.

The stressed feature can be displacement feature, acceleration feature or strain feature. The feature sensor 112 can be Gyro, acceleration sensor or strain sensor, wherein Gyro is used to sense the displacement feature of a corresponding supporting strip component 111, the acceleration sensor is used to sense the acceleration feature of the supporting strip component 111, and the strain sensor is used to sense the strain feature of the supporting strip component 111. According to the sensed stressed feature, the analysis module 12 thus can determine the stress state of the test object 2 (such as position or strength of the force acting on the test object 2). If the test object 2 is a pillow, the analysis module 12 can analyze the sleep quality of a user according to a stress state of the pillow being used by the user.

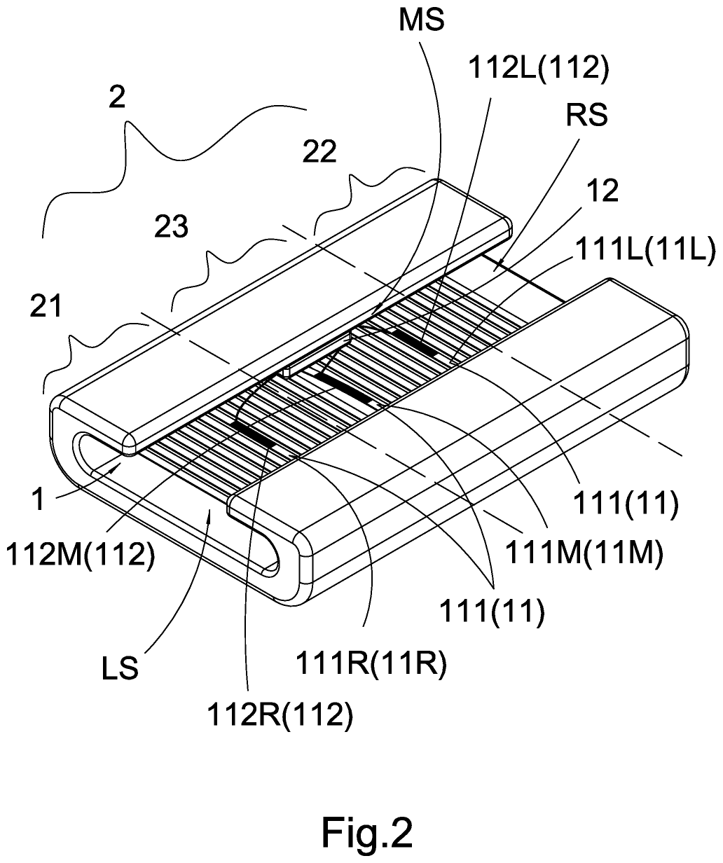

In order to clearly describe the disclosure of the present invention for easy understanding, a strain sensor is depicted in the following as an example of the feature sensor 112. As shown in FIGS. 1 to 2, the test object 2 is defined to have a left area 21, a right area 22 and a middle area 23 thereon, and correspondingly, the stress bearing modules 11 include a left stress bearing module 11L, a right stress bearing module 11R and a middle stress bearing module 11M.

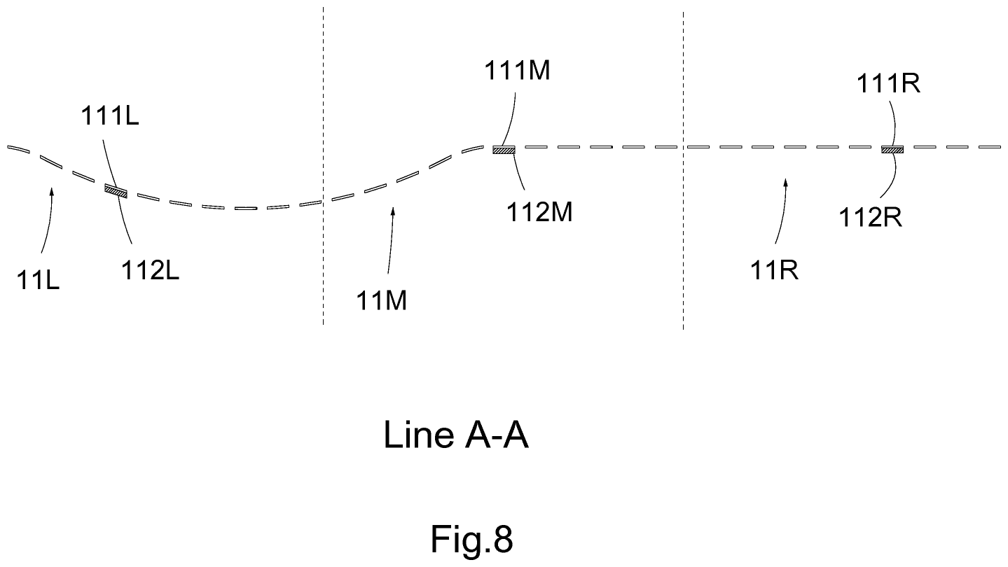

The left stress bearing module 11L is mounted at the left area 21, and includes a left supporting strip component 111L and a left strain sensor 112L. The left supporting strip component 111L provides elastic support for the left area 21, allowing the left supporting strip component 111L to have left elastic deformation shown in FIG. 8 caused by a force exerted on the test object 2. The left strain sensor 112L is used to sense the left elastic deformation.

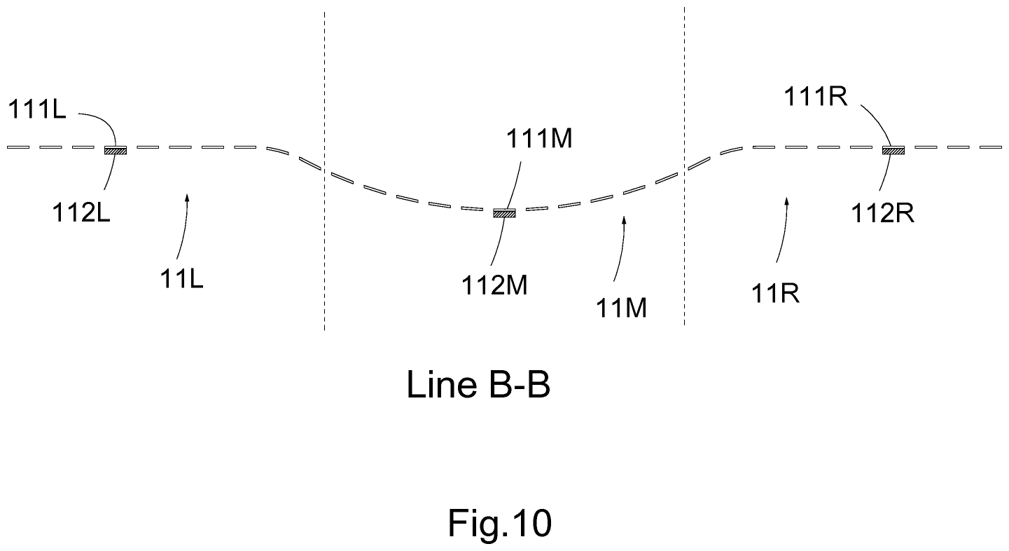

The middle stress bearing module 11M is mounted at the middle area 23, and includes a middle supporting strip component 111M and a middle strain sensor 112M. The middle supporting strip component 111M provides elastic support for the middle area 23, allowing the middle supporting strip component 111M to have middle elastic deformation shown in FIG. 10 caused by a force exerted on the test object 2. The middle strain sensor 112M is used to sense the middle elastic deformation.

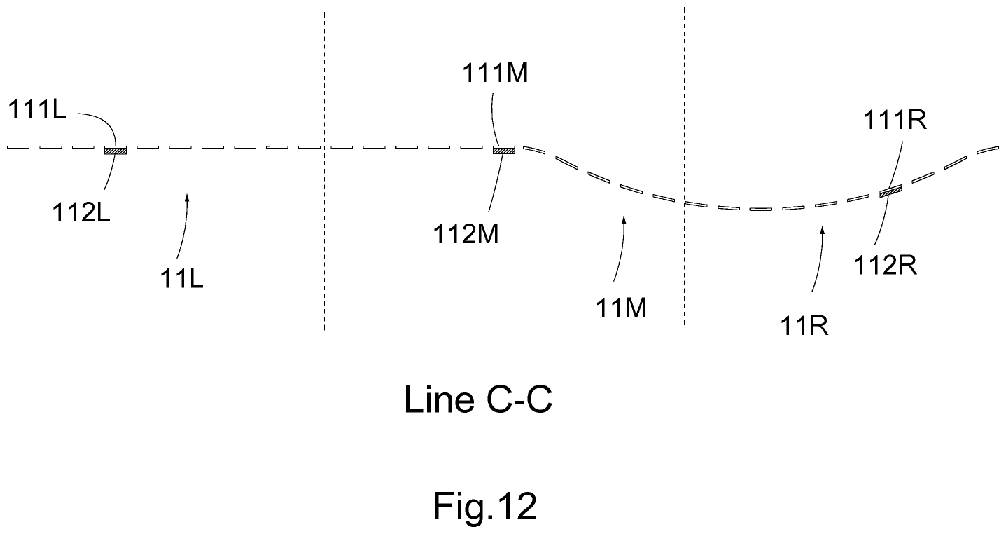

The right stress bearing module 11R is mounted at the right area 22, and includes a right supporting strip component 111R and a right strain sensor 112R. The right supporting strip component 111R provides elastic support for the right area 22, allowing the right supporting strip component 111R to have right elastic deformation shown in FIG. 12 caused by a force exerted on the test object 2. The right strain sensor 112R is used to sense the right elastic deformation.

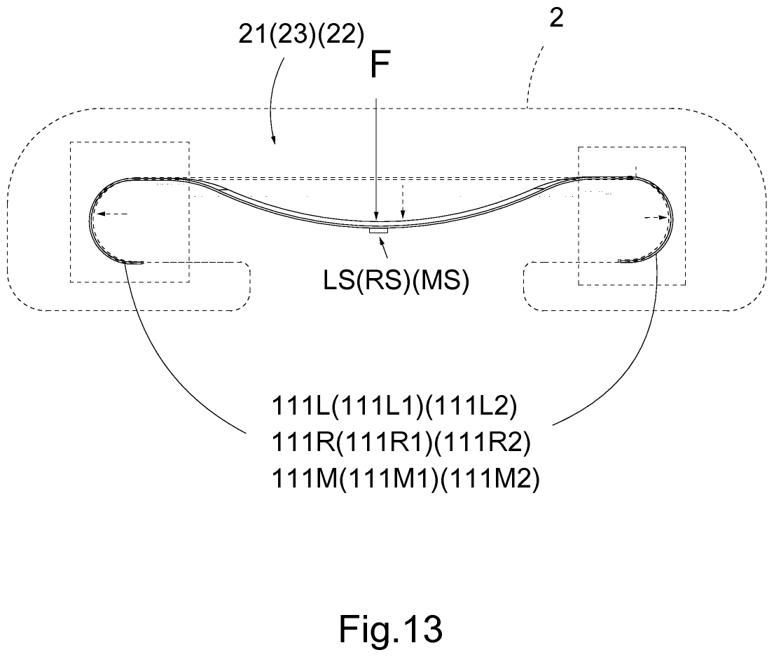

The analysis module 12 conducts a stress analysis for the test object 2 by comparing sensing results from at least two feature sensors 12. As shown in FIG. 2, the analysis module 12 is connected to the left, middle and right strain sensors 112L, 112M, 112R respectively to receive sensing results from those left, middle and right strain sensors 112L, 112M, 112R respectively. When an external force F is exerted on the left area 21 of the test object 2 as shown in FIG. 13, the analysis module 12 analyzes the degree of left elastic deformation shown in FIG. 8 according to the sensing result from the left strain sensor 112L. When the force F is exerted on the middle area 23 of the test object 2 as shown in FIG. 13, the analysis module 12 analyzes the degree of middle elastic deformation shown in FIG. 10 according to the sensing result from the middle strain sensor 112M. When the force F is exerted on the right area 22 of the test object 2 as shown in FIG. 13, the analysis module 12 analyzes the degree of right elastic deformation shown in FIG. 12 according to the sensing result from the right strain sensor 112R. Therefore, the analysis module 12 can analyze a stress state of each of the left, middle and right areas 21, 23, 22 according to the degree of left, middle and right elastic deformation so as to further determine whether the test object 2 is in an expected stressed condition (such as position or strength of the force acting on the test object 2). If the test object 2 is a pillow, the analysis module 12 even can determine a sleep posture of a user who is using the pillow.

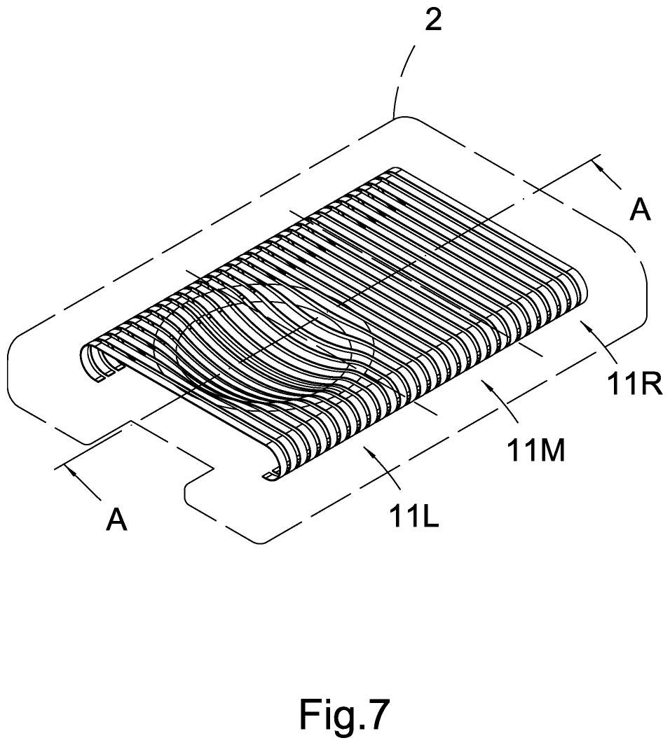

Particularly, as shown in FIG. 7, when the degree of left elastic deformation is respectively greater than the degree of middle elastic deformation and the degree of right elastic deformation, the analysis module 12 can then decide a main position on the test object 2 where the force F is acting is close to the left area 21. If the test object 2 is a pillow, it means its user is having side sleep on a position close to the left area 21 of the pillow.

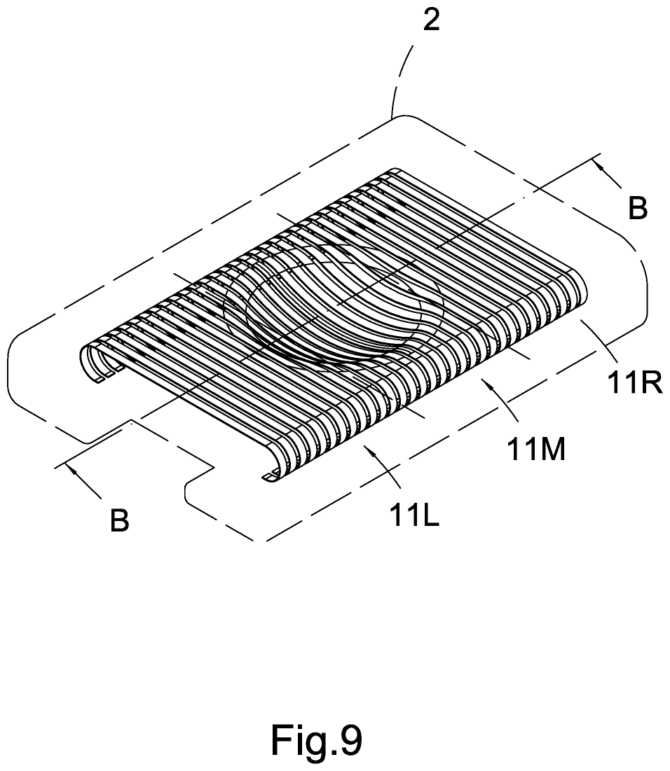

As shown in FIG. 9, when the degree of middle elastic deformation is respectively greater than the degree of right elastic deformation and the degree of left elastic deformation, the analysis module 12 can then decide a main position on the test object 2 where the force F is acting is close to the middle area 23. If the test object 2 is a pillow, it means its user is sleeping face up on a position close to the middle area 23 of the pillow.

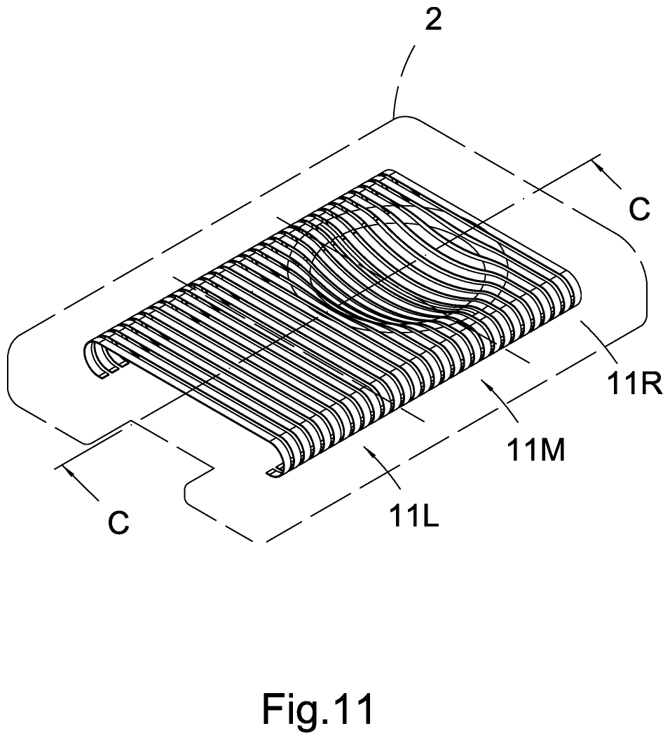

As shown in FIG. 11, when the degree of right elastic deformation is respectively greater than the degree of middle elastic deformation and the degree of left elastic deformation, the analysis module 12 can then decide a main position on the test object 2 where the force F is acting is close to the right area 22. If the test object 2 is a pillow, it means its user is having side sleep on a position close to the right area 22 of the pillow.

It should be noted that, the number of left, middle and right stress bearing modules 11L, 11M, 11R is not limited to one each; alternatively, a plurality of stress bearing modules 11 can be provided at each predetermined area on the test object 2 so as to allow the stress state of each predetermined area to be sensed accurately.

Moreover, at least one of the left, middle and right stress bearing modules 11L, 11M, 11R can be removed in practical use if a reduced number of the stress bearing modules 11 is preferable. The left, middle and right stress bearing modules 11L, 11M, 11R can be made of a composite material composed of carbon fiber and glass fiber or other elastic materials, or can be made of a single material such as carbon fiber, glass fiber or another elastic material, so as to have good structural strength and elastic deformation as expected.

As shown in FIG. 13, the left stress bearing module 111L has a left longitudinal elastic supporting structure 111L1 and a left transverse elastic supporting structure 111L2. The middle stress bearing module 111M has a middle longitudinal elastic supporting structure 111M1 and a middle transverse elastic supporting structure 111M2. The right stress bearing module 111R has a right longitudinal elastic supporting structure 111R1 and a right transverse elastic supporting structure 111R2.

The left longitudinal elastic supporting structure 111L1 provides longitudinal elastic support for the left area 21, allowing the force F acting on the test object 2 to cause left longitudinal elastic deformation of the left longitudinal elastic supporting structure 111L1. The left transverse elastic supporting structure 111L2 provides transverse elastic support for the left area 21, allowing the force F acting on the test object 2 to cause left transverse elastic deformation of the left transverse elastic supporting structure 111L2. The left strain sensor 112L senses the degree of left longitudinal elastic deformation and the degree of left transverse elastic deformation by the left supporting strip component 111L in multiple spatial dimensions, and the analysis module 12 analyzes a longitudinal stress state and a transverse stress state of the left area 21 respectively according to the degree of left longitudinal elastic deformation and the degree of left transverse elastic deformation. Thereby, the stress analysis system 1 of the present invention merely uses a small number of sensors but can accurately analyze the stress state of the left area 21 of the test object 2 in multiple spatial dimensions.

The left longitudinal elastic supporting structure 111L1 can be connected to the left transverse elastic supporting structure 111L2 to form a left space LS. The left space LS is able to receive the left strain sensor 112L or the analysis module 12, and accommodate the left longitudinal elastic deformation of the left longitudinal elastic supporting structure 111L1 or the left transverse elastic deformation of the left transverse elastic supporting structure 111L2. If the test object 2 is a pillow, the left space LS further provides ventilation for the pillow to prevent its user from choke when he or she sleeps face down, and the left longitudinal elastic supporting structure 111L1 and the left transverse elastic supporting structure 111L2 can respectively provide longitudinal elastic support and transverse elastic support for a left part of the pillow.

The middle longitudinal elastic supporting structure 111M1 provides longitudinal elastic support for the middle area 23, allowing the force F acting on the test object 2 to cause middle longitudinal elastic deformation of the middle longitudinal elastic supporting structure 111M1. The middle transverse elastic supporting structure 111M2 provides transverse elastic support for the middle area 23, allowing the force F acting on the test object 2 to cause middle transverse elastic deformation of the middle transverse elastic supporting structure 111M2. The middle strain sensor 112M senses the degree of middle longitudinal elastic deformation and the degree of middle transverse elastic deformation by the middle supporting strip component 111M in multiple spatial dimensions, and the analysis module 12 analyzes a longitudinal stress state and a transverse stress state of the middle area 23 respectively according to the degree of middle longitudinal elastic deformation and the degree of middle transverse elastic deformation. Thereby, the stress analysis system 1 of the present invention merely uses a small number of sensors but can accurately analyze the stress state of the middle area 23 of the test object 2 in multiple spatial dimensions.

The middle longitudinal elastic supporting structure 111M1 can be connected to the middle transverse elastic supporting structure 111M2 to form a middle space MS. The middle space MS is able to receive the middle strain sensor 112M or the analysis module 12, and accommodate the middle longitudinal elastic deformation of the middle longitudinal elastic supporting structure 111M1 or the middle transverse elastic deformation of the middle transverse elastic supporting structure 111M2. If the test object 2 is a pillow, the middle space MS further provides ventilation for the pillow to prevent its user from choke when he or she sleeps face down, and the middle longitudinal elastic supporting structure 111M1 and the middle transverse elastic supporting structure 111M2 can respectively provide longitudinal elastic support and transverse elastic support for a middle part of the pillow.

The right longitudinal elastic supporting structure 111R1 provides longitudinal elastic support for the right area 22, allowing the force F acting on the test object 2 to cause right longitudinal elastic deformation of the right longitudinal elastic supporting structure 111R1. The right transverse elastic supporting structure 111R2 provides transverse elastic support for the right area 22, allowing the force F acting on the test object 2 to cause right transverse elastic deformation of the right transverse elastic supporting structure 111R2. The right strain sensor 112R senses the degree of right longitudinal elastic deformation and the degree of right transverse elastic deformation by the right supporting strip component 111R in multiple spatial dimensions, and the analysis module 12 analyzes a longitudinal stress state and a transverse stress state of the right area 22 respectively according to the degree of right longitudinal elastic deformation and the degree of right transverse elastic deformation. Thereby, the stress analysis system 1 of the present invention merely uses a small number of sensors but can accurately analyze the stress state of the right area 22 of the test object 2 in multiple spatial dimensions.

The right longitudinal elastic supporting structure 111R1 can be connected to the right transverse elastic supporting structure 111R2 to form a right space RS. The right space RS is able to receive the right strain sensor 112R or the analysis module 12, and accommodate the right longitudinal elastic deformation of the right longitudinal elastic supporting structure 111R1 or the right transverse elastic deformation of the right transverse elastic supporting structure 111R2. If the test object 2 is a pillow, the right space RS further provides ventilation for the pillow to prevent its user from choke when he or she sleeps face down, and the right longitudinal elastic supporting structure 111R1 and the right transverse elastic supporting structure 111R2 can respectively provide longitudinal elastic support and transverse elastic support for a right part of the pillow.

In addition, it should be noted that since the feature sensor 112 can be selected as Gyro, acceleration sensor or strain sensor to sense a displacement feature, an acceleration feature or a strain feature respectively for performing stress analysis on a test object 2 to determine a stress state of the test object 2 so as for therapeutic or other purposes. If the test object 2 is a pillow, the analysis module 12 can analyze the sleep quality of a user according to a stress state of the pillow being used by the user.

Therefore, the stress analysis system of the present invention is for performing analysis of a force exerted on a test object, wherein the test object is defined to have a plurality of predetermined areas at different positions thereof. The stress analysis system includes: an analysis module, and at least two stress bearing modules separate from each other, each of which is located at one of the predetermined areas. Each of the stress bearing modules includes a supporting strip component and a feature sensor, wherein the supporting strip component is able to produce a stressed feature in at least one spatial dimension when being subjected to the force, and the feature sensor is able to sense a stress state of the corresponding predetermined area of the test object in at least one spatial dimension, such that the stress analysis system uses a small number of sensors but can accurately analyze the stress state of the test object, thereby solving undesirable problems encountered in the art.

The examples above are only illustrative to explain principles and effects of the invention, but not to limit the invention. It will be apparent to those skilled in the art that modifications and variations can be made without departing from the scope of the invention. Therefore, the protection range of the rights of the invention should be as defined by the appended claims.

What is claimed is:

1. A stress analysis system for performing analysis of a force exerted on a test object, the test object being defined to have at least two predetermined areas at different positions thereof, the stress analysis system including:
at least two stress bearing modules separate from each other, each of which corresponds to one of the at least two predetermined areas on the test object, each of the stress bearing modules including a supporting strip component and a feature sensor, wherein the supporting strip component is able to produce a stressed feature for its corresponding one of the predetermined areas when being subjected to the force, and the feature sensor is able to sense the stressed feature from the supporting strip component to obtain a sensing result; and
an analysis module for obtaining an effect on the supporting strip component from the force exerted on the test object according to the sensing result from the feature sensor of each of the stress bearing modules so as to analyze a stress state of each of the predetermined areas of the test object.

2. The stress analysis system according to claim 1, wherein the feature sensor is Gyro, wherein Gyro is able to sense a displacement feature of the supporting strip component, with the displacement feature used as the stressed feature.

3. The stress analysis system according to claim 1, wherein the feature sensor is an acceleration sensor, wherein the acceleration sensor is able to sense an acceleration feature of the supporting strip component, with the acceleration feature used as the stressed feature; and the strain sensor is able to sense a strain feature of the supporting strip component, with the strain feature used as the stressed feature.

4. The stress analysis system according to claim 1, wherein the feature sensor is a strain sensor, wherein the strain sensor is able to sense a strain feature of the supporting strip component, with the strain feature used as the stressed feature.

5. The stress analysis system according to claim 4, wherein the predetermined areas include a left area and a right area, and the stress bearing modules include a left stress bearing module and a right stress bearing module, wherein,
the left stress bearing module is provided at the left area, and has a left supporting strip component and a left strain sensor, wherein the left supporting strip component provides elastic support for the left area, allowing the force exerted on the test object to cause left elastic deformation of the left supporting strip component, and the left strain sensor is used to sense the left elastic deformation; and
the right stress bearing module is provided at the right area, and has a right supporting strip component and a right strain sensor, wherein the right supporting strip component provides elastic support for the right area, allowing the force exerted on the test object to cause right elastic deformation of the right supporting strip component, and the right strain sensor is used to sense the right elastic deformation;
the analysis module is able to receive sensing results from the left strain sensor and the right strain sensor respectively to obtain the degree of left elastic deformation and the degree of right elastic deformation, and to analyze a stress state of the left area and a stress state of the right area respectively according to the degree of left elastic deformation and the degree of right elastic deformation; wherein,
when the degree of left elastic deformation is greater than the degree of right elastic deformation, the analysis module determines that a main position of the test object being subjected to the force is close to the left area; and
when the degree of right elastic deformation is greater than the degree of left elastic deformation, the analysis module determines that a main position of the test object being subjected to the force is close to the right area.

6. The stress analysis system according to claim 5, wherein the left supporting strip component has a left longitudinal elastic supporting structure and a left transverse elastic supporting structure, wherein the left longitudinal elastic supporting structure provides longitudinal elastic support for the left area, allowing the force exerted on the test object to cause left longitudinal elastic deformation of the left longitudinal elastic supporting structure, and wherein the left transverse elastic supporting structure provides transverse elastic support for the left area, allowing the force exerted on the test object to cause left transverse elastic deformation of the left transverse elastic supporting structure, wherein the left strain sensor senses the degree of left longitudinal elastic deformation and the degree of left transverse elastic deformation by the left supporting strip component, and the analysis module analyzes a longitudinal stress state and a transverse stress state of the left area respectively according to the degree of left longitudinal elastic deformation and the degree of left transverse elastic deformation.

7. The stress analysis system according to claim 6, wherein the left longitudinal elastic supporting structure is connected to the left transverse elastic supporting structure to form a left space, wherein the left space is able to receive the left strain sensor or the analysis module, and accommodate the left longitudinal elastic deformation of the left longitudinal elastic supporting structure or the left transverse elastic deformation of the left transverse elastic supporting structure.

8. The stress analysis system according to claim 7, wherein the test object is a pillow, the left space provides ventilation for the pillow, and the left longitudinal elastic supporting structure and the left transverse elastic supporting structure respectively provide longitudinal elastic support and transverse elastic support for a left part of the pillow.

9. The stress analysis system according to claim 5, wherein the right supporting strip component has a right longitudinal elastic supporting structure and a right transverse elastic supporting structure, wherein the right longitudinal elastic supporting structure provides longitudinal elastic support for the right area, allowing the force exerted on the test object to cause right longitudinal elastic deformation of the right longitudinal elastic supporting structure, and wherein the right transverse elastic supporting structure provides transverse elastic support for the right area, allowing the force exerted on the test object to cause right transverse elastic deformation of the right transverse elastic supporting structure, wherein the right strain sensor senses the degree of right longitudinal elastic deformation and the degree of right transverse elastic deformation by the right supporting strip component, and the analysis module analyzes a longitudinal stress state and a transverse stress state of the right area respectively according to the degree of right longitudinal elastic deformation and the degree of right transverse elastic deformation.

10. The stress analysis system according to claim 9, wherein the right longitudinal elastic supporting structure is connected to the right transverse elastic supporting structure to form a right space, wherein the right space is able to receive the right strain sensor or the analysis module, and accommodate the right longitudinal elastic deformation of the right longitudinal elastic supporting structure or the right transverse elastic deformation of the right transverse elastic supporting structure.

11. The stress analysis system according to claim 10, wherein the test object is a pillow, the right space provides ventilation for the pillow, and the right longitudinal elastic supporting structure and the right transverse elastic supporting structure respectively provide longitudinal elastic support and transverse elastic support for a right part of the pillow.

12. The stress analysis system according to claim 5, wherein the predetermined areas further include a middle area, and the stress bearing modules further include at least one middle stress bearing module, wherein, the middle stress bearing module is provided at the middle area, and has a middle supporting trip component and a middle strain sensor, wherein
the middle supporting strip component provides elastic support for the middle area, allowing the force exerted on the test object to cause middle elastic deformation of the middle supporting strip component, and the middle strain sensor is used to sense the middle elastic deformation;
the analysis module is further able to receive a sensing result from the middle strain sensor to obtain the degree of middle elastic deformation, and to analyze a stress state of the middle area according to the degree of middle elastic deformation, wherein,
when the degree of middle elastic deformation is respectively greater than the degree of right elastic deformation and the degree of left elastic deformation, the analysis module determines that a main position of the test object being subjected to the force is close to the middle area.

13. The stress analysis system according to claim 12, wherein the middle supporting strip component has a middle longitudinal elastic supporting structure and a middle transverse elastic supporting structure, wherein the middle longitudinal elastic supporting structure provides longitudinal elastic support for the middle area, allowing the force exerted on the test object to cause middle longitudinal elastic deformation of the middle longitudinal elastic supporting structure, and wherein the middle transverse elastic supporting structure provides transverse elastic support for the middle area, allowing the force exerted on the test object to cause middle transverse elastic deformation of the middle transverse elastic supporting structure, wherein the middle strain sensor senses the degree of middle longitudinal elastic deformation and the degree of middle transverse elastic deformation by the middle supporting strip component, and the analysis module analyzes a longitudinal stress state and a transverse stress state of the middle area respectively according to the degree of middle longitudinal elastic deformation and the degree of middle transverse elastic deformation.

14. The stress analysis system according to claim 13, wherein the middle longitudinal elastic supporting structure is connected to the middle transverse elastic supporting structure to form a middle space, wherein the middle space is able to receive the middle strain sensor or the analysis module, and accommodate the middle longitudinal elastic deformation of the middle longitudinal elastic supporting structure or the middle transverse elastic deformation of the middle transverse elastic supporting structure.

15. The stress analysis system according to claim 14, wherein the test object is a pillow, the middle space provides ventilation for the pillow, and the middle longitudinal elastic supporting structure and the middle transverse elastic supporting structure respectively provide longitudinal elastic support and transverse elastic support for a middle part of the pillow.

16. The stress analysis system according to claim 12, wherein the middle supporting strip component, the left supporting strip component and the right supporting strip component are each made of a composite material that is selected from a group consisting of carbon fiber and glass fiber.
